(19) United States
(12) Patent Application Publication
(10) Pub. No.: US 2005/0046421 A1
(43) Pub. Date: Mar. 3, 2005

(54) Methods for identifying a compound that can bind to membrane-bound receptors

(76) Inventors: Martyn C. Botfield, Boston, MA (US); Jeffrey Weilee Peng, Granger, IN (US)

Correspondence Address: Nandakumar Govindaswamy;Vertex Pharmaceuticals Incorporated, 130 Waverly Street, Cambridge, MA, 02139, US

(21) Appl. No.: 10/837,558

(22) Filed: Apr. 30, 2004

Related U.S. Application Data
(63) Continuation of application No. PCT/US02/34831, filed on Oct. 30, 2002.
(60) Provisional application No. 60/340,946, filed on Oct. 30, 2001.

(51) Int. Cl. C12P 21/04

(52) U.S. Cl. 324/300

(57) ABSTRACT

The present invention relates to methods of identifying compounds that can bind to a membrane-bound receptor.

### CROSS-REFERENCE TO RELATED APPLICATIONS

The present application is a continuation, and claims the benefit of, International PCT Application No. PCT/US02/34831, filed Oct. 30, 2002, which claims priority to U.S. Provisional Application No. 60/340,946, filed Oct. 30, 2001, the disclosures of both applications being incorporated herein by reference.

### TECHNICAL FIELD OF THE INVENTION

The present invention relates to the use of NMR in combination with virus like particles (VLPs) to study the bound conformation of a known ligand. In particular, the present invention relates to the use of VLPs for the extraction and isolation of membrane-associated receptors for the purpose of identifying potential ligands.

### BACKGROUND OF THE INVENTION

A receptor can be defined as a molecular structure, generally a protein, associated with a biological membrane (i.e. located within or on the surface of a cell membrane), characterized by selective binding of a specific substance—a ligand—where the binding of the ligand initiates either a specific biological response or the transduction of a signal that accompanies the binding. In many cases, structural knowledge of the bound conformation of a receptor with its ligand has allowed scientists to design conformationally-constrained analogs with improved biological properties.

For example, a group at Merck (D F Veber et al, Nature (1981), 292(5818):55-58) was able to design a conformationally-constrained analog of somatastatin, an endogenous peptide which regulates growth. This constraint was designed, based on knowledge of the computationally-determined low-energy conformation. This resulted in a molecule which was both more potent, and more bioavailable. Another group at Smith-Kline empirically developed SKF-38393 etc., a series of conformationally-constrained analogs of dopamine, an endogenous neurotransmitter; these compounds were shown to have selectivity for one subtype of the dopamine receptor (Sidhu A and Kebabian J W, Eur J Pharmacol (1985) 113(3):437-440). Another group at Smith-Kline determined empirically the bound conformation of a peptide which binds the fibrinogen receptor in the clotting cascade, and designed a conformationally-constrained analog two orders of magnitude more potent than the corresponding unconstrained peptide (Samanen J et al, J Med Chem (1991) 34(10):3114-3125). A group at Dupont used the bound conformation of an HIV inhibitor as determined by X-ray to design a series of conformationally-constrained analogs which were exceptionally potent and with high oral bioavailability (Lam P Y et al, Science (1994) 263(5145):380-384). Thus, designing a conformationally-constrained analog is straightforward when the 3D conformation of the ligand as bound to the receptor is known, and the amount of chemical synthesis required to create such analogs with improved biological properties (potency, selectivity, bioavailability) is reduced.

One way of determining the 3D conformation of a ligand as bound to a receptor has been through the use of nuclear magnetic resonance (NMR) spectroscopy. NMR spectroscopy, the process of analyzing a small sample in a uniform magnetic field and obtaining radio frequency data resulting from precisely pulsed radio frequency excitation, was invented by Block and Purcell. NMR spectroscopy has been used for many years in the identification of compounds by comparing the spectra of known compounds with those of the compounds to be analyzed and by providing magnetic parameters (chemical shifts and coupling constants) that have been found to be characteristic of particular types of structures. The techniques employed in this method of spectral analysis are described in the literature, and NMR spectrometers are commercially available. Accordingly, NMR has played a central role in the characterization of the structure and dynamics of proteins, nucleic acids, carbohydrates and their complexes. Second only to crystallography, NMR spectroscopy provides an unparalleled view of structure and it remains second to none in its ability to examine dynamic phenomena. NMR also provides a unique avenue to monitor the full structural and dynamic effects of changes in temperature, solution conditions and the binding of small and large ligands.

Until recently however, the analysis of membrane-associated receptors (i.e. receptors containing membrane-associated proteins, such as intrinsic and transmembrane proteins) and their ligands has long been associated with many problems due to the fact that most of these membrane-associated receptors can only be extracted and/or purified by detergent solubilization. Specifically, although detergent solubilization serves to isolate the membrane-associated receptor, it also results in the removal of the respective membrane-associated receptor from its normal lipid/lipid-protein environment. The disassociation from the membrane-associated receptor's normal lipid/lipid-protein environment usually results in the loss of either partial or complete function of the membrane-associated receptor due to the denaturing effects of the physical removal of the specific membrane protein from its native environment. Thus, the receptor is usually not able to bind its respective ligand after isolation through detergent solubilization. This results in the inability to determine the structural characteristics of the bound conformation of a known ligand bound to its respective membrane-associated receptor.

However, the use of virus like particles in the production of membrane-associated proteins has overcome the problems associated with the extraction of membrane-associated proteins. Virus like particles (VLPs) are self-assembling particles which have a similar physical appearance to virus particles. Specifically, VLPs usually lack or possess dysfunctional copies of certain genes of the wild-type virus, resulting in the VLP being incapable of some function which is characteristic of the wild-type virus (i.e. replication or cell-cell movement). Specifically, VLPs serve to incorporate a protein of interest.

One method of protein incorporation is based upon observations that when one expresses specific structural gene components of retroviruses (the gag gene, or structural protein components from other virus families) as an unprocessed polyprotein in host cells then this gene alone is able to and is responsible for the formation and release of VLPs into the extracellular milieu via a process of budding from the plasma membrane. Most enveloped virus like particles acquire their membrane or “envelope”, a lipid bilayer and associated target proteins, by budding through an appropriate cellular membrane—the plasma membrane in many cases, the ER, Golgi, or nuclear membranes in others. Details of budding processes are known in the prior art (for a general review see e.g. Fields et al. “Fundamental Virology”, Chapter 3, 3r^{d} edition, Lippincott-Raven, 1996). Virus like particles might however also be released from the cell by exocytosis or lysis. Further, methods of making and using VLPs to selectively incorporate or encapsulate membrane-associated proteins is well known in the art (see e.g. WO97/39134 and WO 01/02551; both of which are incorporated herein by reference).

Accordingly, the use of VLP technology allows one skilled in the art to produce a somewhat homogenous sample of a membrane-associated protein without disrupting the protein's membrane-dependent structure or function. Thus, a membrane-associated receptor produced and isolated using VLP technology would be an ideal candidate for determining the 3D bound conformation of its respective ligand.

### SUMMARY OF THE INVENTION

The present invention serves to provide methods of identifying compounds that can bind to a receptor, in general. In particular, the invention relates to methods where the receptor is membrane bound and incorporated into a virus like particle and complexed with a known ligand for the receptor.

Accordingly, in one aspect, the present invention relates to methods of identifying a compound that can bind to a receptor by providing Nuclear Overhauser Effect NMR data or data derived therefrom, on a complex of a receptor bound to a ligand for the receptor, wherein the receptor is incorporated into a virus like particle. The data is then employed, using computational means, to identify a compound that is capable of binding to the receptor through intermolecular interactions. The intermolecular interactions are identified using the Nuclear Overhauser Effect NMR data. Next, the compound is contacted with the receptor under conditions that allow the compound to bind to the receptor so as to determine whether the compound binds to the receptor.

In one embodiment, the data provided is structural data derived from the Nuclear Overhauser Effect NMR data.

In another aspect, the present invention relates to a method of identifying intermolecular interactions between a receptor and a known ligand of the receptor. The receptor is incorporated into a virus-like particle, and contacted with the known ligand, under conditions that allow the formation of a complex between said ligand and said receptor. The complex is the subjected to NMR to generate Nuclear Overhauser Effect data. Optionally, this data can then be converted from the Nuclear Overhauser Effect data to structural coordinate data or to bond angle data. The Nuclear Overhauser Effect data or the converted data can then be interpreted so as to identify intermolecular interactions between the receptor and the ligand in said complex.

The invention also provides a computer for producing a three-dimensional representation of a compound bound to a receptor. Such a computer, appropriately programmed and attached to the necessary viewing device, is capable of displaying a three-dimensional graphical representation of a compound bound to a receptor. Specifically, the computer would comprise a machine-readable data storage medium having a data storage material encoded with machine-readable data, wherein said data comprises Nuclear Overhauser Effect data or data derived therefrom; a working memory for storing instructions for processing said machine-readable data; a central-processing unit coupled to said working memory and to said machine-readable data storage medium for processing said machine readable data into said three-dimensional representation; and a display coupled to said central-processing unit for displaying said three-dimensional representation. The data would be obtained by subjecting a complex comprising a receptor incorporated into a virus-like particle and a compound bound to said receptor to NMR.

The invention also provides a method for evaluating the potential of a chemical entity to associate with a membrane-bound receptor by employing a computational means to perform a fitting operation between the chemical entity and the receptor utilizing Nuclear Overhauser Effect NMR data or data derived therefrom. The Nuclear Overhauser Effect NMR data would be obtained from a complex comprising a receptor incorporated into a virus-like particle and a compound bound to said receptor. The results of said fitting operation can then be analyzed to quantify the association between the chemical entity and the receptor.

In on embodiment, the method described above is performed on a on a series of different chemical entities, of which two or more are selected that have been determined to the potential to bind to the receptor based upon the analysis of the fitting operations, and are capable of being chemically bound to one another directly or through a linker moiety. A compound that comprises the chemical entities selected would then be contacted with the receptor under conditions that allow said compound to bind to said receptor, so that it can be determined whether said compound binds to said receptor.

### DETAILED DESCRIPTION OF THE INVENTION

In order that the invention described herein may be more fully understood, the following detailed description is set forth.

The present invention serves to provide methods of identifying compounds that can bind to a receptor, in general. In particular, the invention relates to methods where the receptor is membrane bound and incorporated into a virus like particle and complexed with a known ligand for the receptor.

A “membrane-bound receptor” is a receptor that is located within or on the surface of a bio-membrane or cell-membrane, such as the plasma membrane in many cases, the ER, Golgi, or nuclear membranes.

“Conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

Accordingly, in one aspect, the present invention relates to methods of identifying a compound that can bind to a receptor by providing NMR data or data derived therefrom, on a complex of a receptor bound to a ligand for the receptor, wherein the receptor is incorporated into a virus like particle. A “virus like particle” (VLP) is a self-assembling particle which has a similar physical appearance to a virus particle, except that it lacks or possesses dysfunctional copies of certain genes of the wild-type virus, resulting in the VLP being incapable of some function which is characteristic of the wild-type virus (i.e. replication or cell-cell movement).

The method to selectively incorporate or encapsulate a protein of interest into virus like particles is well known in the art, and is based upon observations that when one expresses specific structural gene components of retroviruses (the gag gene, or structural protein components from other virus families) as an unprocessed protein in host cells then this gene alone is able to and is responsible for the formation and release of VLPs into the extracellular milieu via a process of budding from the plasma membrane. This observation has been adapted and developed into a methodology in which peptides or polypeptides are incorporated selectively into or encapsulated within host cell derived defined vesicular particles. Techniques to isolate or extract a receptor of interest using VLPs are well known in the art. For example, WO 01/02551 and WO 97/39134, both of which are herein incorporated by reference in their entirety, describe various methods to produce VLPs containing a protein of interest, and their use in screening for drug candidates, as well as their use in functional genomic applications.

Specifically, WO 01/02551 describes methods of making VLPs containing a protein of interest using a generic tagging strategy. For instance, the receptor of interest is usually heterologous to the virus like particle, and can be engineered, by methods well known in the art, to comprise at least a fragment of a virus capsid or envelope protein, or a precursor of a virus capsid or envelope protein. It might however also comprise at least a fragment of a capsid or envelope protein of a virus like particle, or a precursor of said capsid or envelope protein.

Retroviruses have a protein capsid which contains among other constituents the viral genetic material and the reverse transcriptase complex. Capsid or envelope proteins might be chosen from a variety of virus families including, but not limited to, retroviruses, picornaviruses, reoviruses, polyomaviruses, papillomaviruses, parvoviruses, nodaviruses, coronaviruses, herpesviruses, hepadnaviruses, baculoviruses and bacteriophages. A list of particularly suitable viruses is given in Table 1 of the WO 01/02551 patent application.

Outside the capsid is a lipid bilayer derived from the host cell plasma membrane in which viral envelope glycoproteins are embedded. During the infection cycle these envelope glycoproteins initiate an infection by recognizing and binding specific receptors on the surface of a host cell and inducing fusion of the viral and cell membranes. After intracellular genome replication and its integration into the cell chromosome, viral RNAs encoding structural proteins are produced and nascent virions are assembled. Newly synthesized viral capsids specifically incorporate viral glycoproteins from the plasma membrane during viral budding while, for the most part, excluding the cellular proteins. This retroviral assembly process is an important aspect of the basic molecular biology of retroviruses. The complexity of this process of viral capsid formation and release from the host cell by the budding process is described in more detail below.

The genome of all retroviruses codes for principally three major gene products, notably the gag gene coding for structural proteins, the pol gene coding for reverse transcriptase and associated proteolytic polypeptides, nuclease and integrase associated functions, and env whose encoded glycoprotein membrane proteins are detected on the surface of infected cells and also on the surface of mature released virus particles. The gag gene of all retroviruses analyzed so far have an overall structural similarity and are conserved particularly at the amino acid level within each group.

The gag and the pol genes can be grouped together for both products and are synthesized as a simple high molecular weight precursor polyprotein e.g. Pr65^{`9} (for the Murine leukaemia virus, MuLV) or Pr2OO^{G}a⁹⁻Pol which is subsequently cleaved to give rise to the mature proteins. The Gag proteins give rise to the core proteins excluding the reverse transcriptase. For Mul-V the Gag precursor polyprotein is Pr65^{Ga9} and is cleaved into four proteins, and it appears that these cleavages are mediated by a viral protease. The Mul-V Gag protein exists in a glycosylated and a non-glycosylated form. The glycosylated forms are cleaved from gPr80^{(;a9} which is synthesized from a different inframe initiation codon located upstream from the AUG codon for the non-glycosylated Pr65^{G}a⁹. Deletion mutants of MuLV that do not synthesize the glycosylated Gag are still infectious, thus raising the question over the importance of the glycosylation events. The post translational cleavage of the HIV-1 Gag precursor of 55 000 Da (pr55^{G}a⁹) by the virus coded protease yields the N-myristoylated and internally phosphorylated p17 matrix protein (p17MA), the phosphorylated p24 capsid protein (p24CA), and the nucleocapsid protein p15 (p15NC), which is further cleaved into p9 and p6. Translation of the MuLV pol gene is achieved by a ribosomal −1 frame shift close to the end of the gag gene. The translation frame shift allows the synthesis of a 160 kD poly-protein consisting of a truncated Gag fusion protein fused to the product of the pol reading frame. However, the level of the GagPol fusion protein production is only 5-10% of the level of production of Gag protein (Jacks et al., Cell 55, 447-458, 1988; Wilson et al., Cell 55, 11591169, 1988).

The pol gene encodes the viral enzyme protease, reverse transcriptase, and integrase which are cleaved from the precursor by the viral protease (Lightfoote et al., J. Virol. 60, 771-775, 1986; Oroszlan and Luftig Curr Top Microbiol Immunol 157, 153-185,1990; Peng et al., J. Virol. 65, 2751-2756, 1991).

The env gene encodes the surface glycoproteins of the virion that are necessary for initiating an infection cycle. Although not closely related to one another the env genes of different groups show a great deal of structural similarity. The amino terminal sequence of the env product encodes a signal peptide which is cleaved off as a consequence of transmembrane processing of the Env precursor. The env gene product of MuLV Pr90^{E}″″ is glycosylated and cleaved to gp70 and p15E, which remain bound to each other via a disulphide linkage. P15E is a transmembrane protein with its carboxyl terminus located internal to the lipid membrane and its amino terminus located external to the membrane. In electron micrographs p15E represents the spikes on the viral envelope while the gp70 is the knob that surmounts the spike. As already described the larger amino terminal protein contains determinants to specify host range. The smaller carboxyl terminal protein always contains, near its carboxyl terminus a hydrophobic domain of 20 amino acids or more, constituting a transmembrane anchor region, followed by a basic amino acid and a cytoplasmic domain of varying size, which is presumably involved in the recognition of capsid proteins.

Assembly of retroviruses takes place by a budding process at the cellular plasma membrane. Studies with several retroviruses have demonstrated that the Gag poly-protein expressed in the absence of other viral components is self sufficient for particle formation and budding at the cell surface (Wills and Craven AIDS 5, 639-654, 1991; Zhou et al., J. Virol. 68, 2556-2569, 1994; Morikawa et al., Virology 183, 288-297, 1991; Royer et al., Virology 184, 417422, 1991; Gheysen et al., Cell 59, 103-112, 1989; Hughes et al., Virology 193, 242-255, 1993; Yamshchikov et al., Virology 214, 50-58, 1995). Formation of retrovirus like particles upon expression of the Gag precursor in insect cells using a Baculovirus vector has been demonstrated by several groups (Delchambre et al., EMBO J. 8, 2653-2660, 1989; Luo et al., Virology 179, 874-880, 1990; Royer et al., Virology 184, 417-422, 1991; Morikawa et al., Virology 183, 288-297, 1991; Zhou et al., J. Virol. 68, 2556-2569, 1994; Gheysen et al., Cell 59, 103-112, 1989; Hughes et al., Virology 193, 242-255, 1993; Yamshchikov et al., Virology 214, 50-58, 1995).

Furthermore, it has been reported that the amino terminal region of the Gag precursor is a targeting signal for transport to the cell surface and membrane binding which is required for virus assembly (Yu et al., J. Virol. 66, 4966-4971, 1992; an, X et al., J. Virol. 67, 6387-6394, 1993; Zhou et al., J. Virol: 68, 25562569, 1994; Lee and Linial J. Virol. 68, 6644-6654, 1994; Dorfman et al., J. Virol. 68, 1689-1696, 1994; Facke et al., J. Virol. 67, 4972-4980, 1993). The mechanism of specific incorporation of envelope protein into the plasma membrane derived envelope of the virus particles is not understood, but interaction of Env with the matrix protein seems to be important (Yu et al., J. Virol. 66, 4966-4971, 1992; Dorfman et al., J. Virol. 68, 1689-1696, 1994; Gallaher et al., AIDS Res Hum Retroviruses 11, 191-202, 1995; Bugelski, P. J. et al., AIDS Res Hum Retroviruses 11, 55-64, 1995).

Once a receptor of interest in incorporated within a VLP to form a sample, the VLP sample is contacted with a known ligand under conditions which allow the formation of a ligand-receptor complex. A “known ligand” is a compound or substance that is known in the art to bind to the receptor of interest, and where the binding of the ligand initiates either a specific biological response or the transduction of a signal that accompanies the binding. The term “ligand-receptor complex” refers to a molecular complex formed by associating a receptor of interest with a known ligand or chemical entity, for example, a potential ligand.

The ligand-receptor complex is then subjected to NMR to generate Nuclear Overhauser Effect data. The techniques employed in NMR are described in the literature, and NMR spectrometers are commercially available. For example, the archetypal NMR experiment to generate NMR data to be used according to the present invention is the NOESY (Nuclear Overhauser Effect SpectroscopY). The NOESY allows one to identify spatially proximal proton pairs (within ˜<=5 Angstroms distance) within a given molecule. NOESY can be performed via a wide variety of NMR pulse sequences. In its most common incarnation, the NOESY provides a two-dimensional NMR spectrum. The spectrum is most often depicted as a 2D square matrix contour plot, in which “spots” or “peaks” of varying intensity appear at various diagonal and off-diagonal positions. The diagonal peaks correspond to the NMR resonances belonging to the various protons of the molecule, i.e. each resolved diagonal peak corresponds to a particular proton. The desired inter-proton distance information lies in the cross peaks that connect the diagonal peaks. Specifically, the appearance of an NOE cross peak between two proton diagonal peaks means that the corresponding protons are spatially close, i.e. within ˜5 angstroms. In the literature, the NOESY cross peaks are often referred to as simply NOEs; this convention will be adopted hereafter. The NOE intensities build up during a parametric delay within the NOES; the delay is known as the “mixing time”. If the NOESY is measured for short enough mixing times, then the relative intensities of the NOEs correlate with relative inter-proton distances. More rigorously, the build-up rates of the NOEs during the mixing time will be proportional to the inverse sixth power of the inter-proton distances.

It is important to appreciate the effects of molecular mass on the NOE cross peak behavior. Large molecules, such as GPCR's tumble slowly in solution. As a result, the NOEs are positive with respect to the diagonal peaks. Additionally, the NOEs are intense and build up rapidly during the NOESY mixing time. In contrast, small molecules (e.g. Mᵣ<1000), tumble rapidly in solution. As a result, the NOE build-up rates are much slower. Moreover, the NOEs are often of opposite sign relative to the diagonal peaks, zero, or only weakly positive. Thus, for short mixing times (<200 ms), small molecule NOEs are expected to be much weaker than those from the large molecules.

After measuring a NOESY, the spectroscopist must assign as many NOEs possible to specific protons pairs within the molecule. The ability to assign the NOEs assumes that the proton resonances have already been assigned via standard NMR experiments. After assignment, one has a database of interproton distance restraints. It is this database of assigned distance restraints that is the input to standard algorithms available from commercial vendors to produce three-dimensional solution structures that are consistent with the NOE data.

Molecules that tumble rapidly in solution produce sharp, intense NMR resonance lines. In contrast, molecules that tumble more slowly give broader NMR resonance lines which compromise the sensitivity and accuracy of all NMR experiments. As a result, NOESY spectra of large target molecules such as membrane receptors and ion-channels are difficult to interpret, thus hinder structure determination.

Under certain circumstances, the above complications can be bypassed by using the so-called “transferred NOE” (tNOE) method. This method works for ligands that undergo rapid chemical exchange between the free and target-bound states. The conditions conducive to fast exchange can be described in terms of the equilibrium dissociation constant K_{D} that pertains to the ligand binding equilibrium. Ligands that bind with K_{D}>100 nM are typically in fast exchange. A consequence of fast exchange is that a single set of NMR resonances are observed for the ligand. In the tNOE method, one exploits the aforementioned differential behavior of the NOESY experiment for large molecules versus small molecules. Specifically, while the ligand is bound to the target molecule, it transiently develops NOEs characteristic of the bound state conformation. Additionally, because the bound ligand shares the slow tumbling of the much larger target, the bound state NOEs are positive with respect to the diagonal and build up rapidly during the NOESY mixing time. The bound state NOEs are then transferred to the free state via chemical exchange (the ligand leaves the target) and become the transferred NOEs. Once in the free state, the lone ligand tumbles rapidly, yielding the characteristically sharp and intense signals. In this manner, the bound state NOEs (the tNOEs) can be easily and sensitively detected via the sharp resonances of the free ligand. To capitalize on the desirable properties of the ligand resonances and to foster selective observation of the ligand signals, one typically works with a molar excess of ligand over target in the range of 10-100:1. The final result is a NOESY spectrum in which the diagonal peaks primarily contain contributions from the ligand free state, while the cross-peaks mainly contain contributions from the tNOEs. There is also a contribution from NOEs native to the free ligand state. However, as stated above, the free state NOEs are expected to be much weaker than the tNOEs. Proper analysis of the NOESY cross peaks leads to the determination of the bound ligand conformation. For review, see e.g. Kisselev et al., Proc. Natl. Acad. Sci., Vol. 95, pp.4270-4275 (1998); see also, Inooka et al., Nature Struct. Biol., Vol. 8, pp. 181-164 (2001).

It should also be noted that other complementary NMR experiments are available for yielding structural information of the bound ligand. For example, one can use transferred cross-correlated relaxation measurements (Carlomagno, T., Felli, I. C., Czech, M., Fischer, R., Sprinzl, M., and Griesinger, C., “Application to the Determination of Sugar Pucker in an aminacylated tRNA-Mimetic Weakly Bound to EF-Tu”, J. Am. Chem. Soc. 121, 1945-1948 (1999)), changes in relaxation anisotropy (Tjandra, N., Garrett, D. S., Gronenborn, A. M., Bax, A. and Clore, G. M., “Defining long range order on NMR structure determination from the dependence of heteronuclear relaxation times on rotational diffusion anistropy”, Nat Struct Biol 4(6), 443-449 (1997)), transferred residual dipolar couplings in anisotropic media (Koenig, B. W., Mitchell, D. C., Konig, S., Grzesiek, S., Litman, B. J., and Bax, A., “Measurement of dipolar couplings in a transducin peptide fragment weakly bound to photoactivated rhodopsin”, J. Biolmol. NMR. 16(2), 121-125 (2000)), and potentially even the use of spin labels (“Spin Label Enhanced NMR Screening”, Jahnke, W., Rudisser, S., and Zurini, M., J. Am. Chem. Soc. 123, 3149-3150 (2001)). Presently, these methods are not as sensitive or as straightforward as the NOESY. However, on-going improvements in NMR technology and isotope-labeling strategies may change this.

Optionally, the Nuclear Overhauser Effect NMR data can be converted into structural coordinate data or bond angle data. The term “structure coordinates” refers to Cartesian coordinates derived from mathematical equations related to the patterns obtained on diffraction of a monochromatic beam of X-rays by the atoms (scattering centers) of a protein or protein complex in crystal form. The diffraction data are used to calculate an electron density map of the repeating unit of the crystal. The electron density maps are then used to establish the positions of the individual atoms of the enzyme or enzyme complex.

NMR structure determination algorithms generate an ensemble of Cartesian (x,y,z) coordinates of the molecule that best satisfy the collection of experimentally observed conformational constraints. These constraints are most commonly derived from Nuclear Overhauser Effect NMR data, but may include other types of data as well (see above). In the most common approach, the constraints are vast numbers of close inter-proton distances from multi-dimensional NOESY spectra. Distance geometry programs such as DGII (part of NMRchitect, Biosym/Molecular Simulations Inc., San Diego Calif. Original work described in Havel, T. F. “The Sampling Properties of Some distance Geometry Algorithms Applied to Unconstrained Polypeptide Chains: A Study of 1830 Independently Computed Conformations”, Biopolymers, 29 1565 (1985)) then searches for sets of Cartesian coordinates that best satisfy these inter-proton distance constraints. The result are a set of structures consistent with the Nuclear Overhauser Effect NMR data. The structures are then further refined with respect to known physical properties of biomolecules (e.g. energy minimization via molecular dynamics simulations to avoid bad intramolecular contacts). One should keep in mind that while Nuclear Overhauser Effect data is the most common experimental observable, commercial software programs are available that can convert any one of the types of NMR structural data mentioned above into molecular structures (e.g. in Cartesian coordinates) consistent with the experimental data. The general manner of obtaining these structure coordinates, interpretation of the coordinates and their utility in understanding the protein structure, as described herein, will be understood by those of skill in the art and by reference to standard references such as:
1. Neuhaus, D.; Williamson M. P. The Nuclear Overhauser Effect in Structural and Conformational Analysis; VCH Publishers, Inc.; New York (1989).
2. Nilges, M; Clore, G. M; Gronenborn, A. M. “Determination of Three-Dimensional Structures of Proteins From Interproton Distance Data by Dynamical Simulated Annealing From a Random Array of Atoms”, FEBS Lett 239, 129-136 (1988).
3. Havel, T. F. “An Evaluation of Computational Strategies for Use in the Determination of Protein Structure from Distance Constraints obtained by Nuclear Magnetic Resonance”, Prog Biophys Mol Biol 56 43-78 (1991).
4. User's Guide for “NMRchtect 95.0” BIOSYM/Molecular Simulations Inc., San Diego Calif.

The following abbreviations are used to describe structure coordinates that can be generated from the NMR analysis on the receptor-ligand complex.

“Atom type” refers to the element whose coordinates are measured. The first letter in the column defines the element.

“X, Y, Z” define the atomic position of the element measured.

In describing protein structure and function, reference is made to amino acids comprising the protein. The amino acids may also be referred to by their conventional abbreviations, as shown in the table below.

| | | | | | |
|---|---|---|---|---|---|
| A= | Ala= | Alanine | T= | Thr= | Threonine |
| V= | Val= | Valine | C= | Cys= | Cysteine |
| L= | Leu= | Leucine | Y= | Tyr= | Tyrosine |
| I= | Ile= | Isoleucine | N= | Asn= | Asparagine |
| P= | Pro= | Proline | Q= | Gln= | Glutamine |
| F= | Phe= | Phenylalanine | D= | Asp= | Aspartic Acid |
| W= | Trp= | Tryptophan | E= | Glu= | Glutamic Acid |
| M= | Met= | Methionine | K= | Lys= | Lysine |
| G= | Gly= | Glycine | R= | Arg= | Arginine |
| S= | Ser= | Serine | H= | His= | Histidine |

The manner of obtaining these structure coordinates, interpretation of the coordinates and their utility in understanding the protein structure, as described herein, will be understood by those of skill in the art and by reference to standard texts such as Crystal Structure Analysis, Jenny Pickworth Glusker and Kenneth N. Trueblood, 2nd Ed. Oxford University Press, 1985, New York; and Principles of Protein Structure, G. E. Schulz and R. H. Schirmer, Springer-Verlag, 1985, New York.

Those of skill in the art understand that a set of structure coordinates for a bound ligand is a relative set of points that define a shape in three dimensions. Thus, it is possible that an entirely different set of coordinates could define a similar or identical shape. Moreover, slight variations in the individual coordinates will have little effect on overall shape. However, in terms of the binding pockets of the receptor of interest, these variations would not be expected to significantly alter the nature of ligands that could associate with those pockets.

These variations in coordinates may be generated because of mathematical manipulations of the ligand's bound structure coordinates. For example, the structure coordinates could be manipulated by fractionalization of the structure coordinates, integer additions or subtractions to sets of the structure coordinates, inversion of the structure coordinates or any combination of the above.

Alternatively, modifications due to mutations, additions, substitutions, and/or deletions of amino acids, or other changes could also account for variations in structure coordinates. If such variations are within an acceptable standard error as compared to the original coordinates, the resulting three-dimensional shape is considered to be the same. Thus, for example, a ligand that bound to the active site binding pocket of a known receptor of interest would also be expected to bind to another binding pocket whose structure coordinates defined a shape that fell within the acceptable error.

Various computational analyses may be used to determine whether a potential ligand is sufficiently similar to the known ligand which bound to the receptor of interest. Such analyses may be carried out in well known software applications, such as the Molecular Similarity application of QUANTA (Molecular Simulations Inc., San Diego, Calif.) version 4.1, and as described in the accompanying User's Guide.

According to an alternate embodiment, this invention provides a computer for producing a three-dimensional representation of a compound bound to a membrane-bound receptor, wherein said computer comprises:
(a) a machine-readable data storage medium comprising a data storage material encoded with machine-readable data, wherein said data comprises Nuclear Overhauser Effect data or data derived therefrom obtained by subjecting a complex comprising a receptor incorporated into a virus-like particle and a compound bound to said receptor to NMR;
(b) a working memory for storing instructions for processing said machine-readable data;
(c) a central-processing unit coupled to said working memory and to said machine-readable data storage medium for processing said machine readable data into said three-dimensional representation; and
(d) a display coupled to said central-processing unit for displaying said three-dimensional representation.

As mentioned above, the coordinate data generated from the NMR analysis of the receptor-ligand complex coordinate data is useful for screening and identifying other potential ligands of the receptor of interest. For example, the structure encoded by the data may be computationally evaluated for its ability to associate with a receptor of interest or to design and/or generate other potential ligands. Such compounds that associate with the receptor of interest may inhibit the receptor from binding its respective ligand, and are potential drug candidates. Additionally or alternatively, the structure encoded by the data may be displayed in a graphical three-dimensional representation on a computer screen. This allows visual inspection of the structure, as well as visual inspection of the structure's association with the compounds.

One skilled in the art may use one of several methods to screen compounds for their ligand potential. This process may begin by visual inspection of, for example, the bound conformation of the ligand on the computer screen based on the structure coordinates generated by the NMR analysis or other coordinates which define a similar shape generated from the machine-readable storage medium. Selected compounds may then be positioned in a variety of orientations, or docked, within the receptor of interest. Docking may be accomplished using software such as Quanta and Sybyl, followed by energy minimization and molecular dynamics with standard molecular mechanics force fields, such as CHARMM and AMBER.

Specialized computer programs may also assist in the process of selecting compounds as potential ligands. These include:
1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK.
2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif.
3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.
4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

According to another embodiment, the invention provides a method for evaluating the potential of a chemical entity to associate with a membrane-bound receptor. The term “chemical entity”, as used herein, refers to chemical compounds, complexes of at least two chemical compounds, and fragments of such compounds or complexes. The term “association between” or “associating with” refers to a condition of proximity between a chemical entity or compound, or portions thereof, and a binding pocket or binding site on a protein. The association may be non-covalent—wherein the juxtaposition is energetically favored by hydrogen bonding or van der Waals or electrostatic interactions—or it may be covalent. Computational means can then be used to perform a fitting operation between the chemical entity and the receptor using the NMR data generated as described above.

In an alternate embodiment, a series of different chemical entities can be analyzed. Two or more of the entities that have the potential to bind to the receptor (based on the fitting operations described above) and are capable of being chemically bound to one another directly or through a linker moiety can then be selected.

If two or more chemical entities are selected, they can be designed or assembled into a single compound or complex. Assembly may be preceded by visual inspection of the relationship of the fragments to each other on the three-dimensional image displayed on a computer screen in relation to the structure coordinates of P38γ. This would be followed by manual model building using software such as Quanta or Sybyl [Tripos Associates, St. Louis, Mo.].

Useful programs to aid one of skill in the art in connecting the individual chemical entities or fragments include:
1. CAVEAT (P. A. Bartlett et al, “CAVEAT: A Program to Facilitate the Structure-Derived Design of Biologically Active Molecules”, in Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp. 51-66 (1994)). CAVEAT is available from the University of California, Berkeley, Calif.
2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992).
3. HOOK (M. B. Eisen et al, “HOOK: A Program for Finding Novel Molecular Architectures that Satisfy the Chemical and Steric Requirements of a Macromolecule Binding Site”, Proteins: Struct., Funct., Genet., 19, pp. 199-221 (1994). HOOK is available from Molecular Simulations, San Diego, Calif.

Instead of proceeding to build an potential ligand of a receptor of interest in a step-wise fashion one as described above, potential ligands may be designed as a whole or “de novo” using either an unbound receptor of interest or optionally including some portion(s) of a known ligand. There are many de novo ligand design methods including:
1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.
2. LEGEND (Y. Nishibata et al., Tetrahedron, 47, p. 8985 (1991)). LEGEND is available from Molecular Simulations Incorporated, San Diego, Calif.
3. LeapFrog (available from Tripos Associates, St. Louis, Mo.).
4. SPROUT (V. Gillet et al, “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention [see, e.g., Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)].

Once a compound that might be a potential ligand has been designed or selected by the above methods, the efficiency with which that potential ligand may bind to a receptor of interest may be tested and optimized by computational evaluation. For example, a potential ligand must preferably demonstrate a relatively small difference in energy between its bound and free states (i.e., a small deformation energy of binding). Thus, the most efficient potential ligands should preferably be designed with a deformation energy of binding of not greater than about 10 kcal/mole, more preferably, not greater than 7 kcal/mole.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. ©1995); AMBER, version 4.1 (P. A. Kollman, University of California at San Francisco, ©1995); QUANTA/CHARMM (Molecular Simulations, Inc., San Diego, Calif. ©1995); Insight II/Discover (Molecular Simulations, Inc., San Diego, Calif. ©1995); DelPhi (Molecular Simulations, Inc., San Diego, Calif. ©1995); and AMSOL (Quantum Chemistry Program Exchange, Indiana University). These programs may be implemented, for instance, using a Silicon Graphics workstation such as an Indigo² with “IMPACT” graphics. Other hardware systems and software packages will be known to those skilled in the art.

Another approach enabled by this invention, is the computational screening of small molecule databases for potential ligands that can bind in whole, or in part, to a receptor of interest. In this screening, the quality of fit of such potential ligands may be judged either by shape complementarity or by estimated interaction energy [E. C. Meng et al., J. Comp. Chem., 13, 505-524 (1992)].

Once potential compounds are identified, these compounds should be tested for their ability to bind to the receptor. Thus, the receptor would be contacted with the potential compounds under conditions that would allow for binding to the receptor. The determination of binding (or lack of binding) can be performed by numerous assays well-known in the art. Specifically, WO97/39134 and WO 01/02551, both of which are incorporated herein by reference, describe assays for detecting a compounds ability to bind to a receptor incorporated into a VLP.

While we have described a number of embodiments of this invention, it is apparent that our basic constructions may be altered to provide other embodiments which utilize the products, processes and methods of this invention. Therefore, it will be appreciated that the scope of this invention is to be defined by the appended claims, rather than by the specific embodiments which have been presented by way of example.

### EXAMPLES

### Example 1

Preliminary Experiments

The receptor of interest, as incorporated within a VLP, is prepared according to well-known techniques disclosed and/or cited to above, and subsequently isolated into a homogeneous sample. The receptor sample is then incubated with a ligand known to bind to the receptor of interest. Concurrently, preliminary experiments conducted on the known ligand are needed to assign the ligand resonances. These experiments are well known in the art, and can be performed using well known NMR techniques. Specifically, one records preliminary experiments on the ligand/receptor sample to assign the ligand proton resonances. This will then facilitate assignment of tNOEs in the subsequent steps. The preliminary experiments typically include those relying on scalar coupling information, such as COSY and TOCSY, and again, are well known by one skilled in the art. For review, see e.g. Kisselev et al., Proc. Natl. Acad. Sci., Vol. 95, pp.4270-4275 (1998); see also, Inooka et al., Nature Struct. Biol., Vol. 8, pp. 181-164 (2001).

NOESY Experiments of Ligand Bound to the Receptor of Interest

One measures a NOESY for the ligand/target sample by methods described and well known in the art. The ligand should be in molar excess of the receptor of interest, as incorporated into a VLP, by a factor approximately 10. On account of the ligand binding to the receptor of interest, one expects to observe t-NOEs that correspond to the bound conformation of the ligand. For example, in polypeptide ligands, these will include medium and long-range NOEs (medium, long range means between residues that are not sequential in the polypeptide chain) that are not present in the free state (i.e. the free state does not have a fixed conformation). The identification of tNOEs are facilitated by comparison to reference NOESY spectra described below.

Reference NOESY Experiments

The NOESY data generated above contains potential contributions from the ligand in its free state. This free state contribution should be checked for and if significant, subtracted from the NOESY spectrum in part 1. To this end, two control experiments may be performed, depending on the available information. Specifically, if one has access to a known ligand which binds tighter then the ligand currently being used to bind to the receptor of interest, then small amounts of this “tighter-binder” ligand can be added to the previous ligand/receptor sample. If the “tighter-binder” ligand competes with the same binding site as the known first ligand, it will be displaced by the tighter binder ligand. A NOESY experiment then reveals just the free ligand NOEs and also verifies that the ligand competes for the binding site of a known ligand.

On the other hand, a known “tighter-binder” ligand may not be available. Or, the ligand binds to the target but not at the same place as the known binder. In this case, one must prepare a second NMR control sample that consists as nearly as possible of the same concentration the known ligand as in the ligand/receptor sample under identical buffer conditions. Again, one records a NOESY and the resulting NOE cross peaks yield just the free state contributions. It should be noted that typical lead compounds have rather low molecular weights (Mᵣ<=1000). For such low molecular weight compounds, the free state NOE cross peaks often have magnitudes dramatically smaller than those of the bound state, and may even be fortuitously zero. This obviously simplifies the interpretation of the NOE spectrum.

However, the NOESY experiment represents only one source of structural information. In principle, it is possible to use any other experiment that also produces exchange-averaged conformational information. Examples include cross-correlation relaxation experiments, scalar and residual dipolar coupling experiments, anisotropy experiments. At the moment, the NOESY experiment is perhaps the most sensitive and straightforward experiments with a high return of structural information relative the work required for setup. However, given the advent of cold probes, this may cease to be true in the future.

Structure Calculations

The free state contribution to the NOESY, as described above, is subtracted resulting in a NOESY spectrum that contains purely information regarding the bound conformation of the known ligand. These NOE's are assigned and quantified using standard techniques. For example, cross peak volumes can be measured using commercial software to classify NOE's as strong, medium and weak. The NOE's are quantified using standard techniques (e.g. peak volume integration) and subjected to standard solution-state NMR structure-determination algorithms. Examples of the such algorithms include the combined distance-geometry/simulated annealing methods available from commercial vendors. The results are an ensemble of conformations that are consistent with the tNOE data. These results can be converted into structure coordinates, by methods described above. The structure coordinates can then be used to identify other potential ligands for the receptor of interest.

1. A method of identifying a compound that can bind to a membrane-bound receptor comprising the steps of:
a) providing Nuclear Overhauser Effect NMR data or data derived therefrom on a complex of a receptor bound to a known ligand for said receptor, wherein said receptor is incorporated into a virus-like particle;
b) employing said data together with computational means to identify a compound capable of binding to said receptor through intermolecular interactions identified between said receptor and said known ligand of the receptor using the data provided in step a);
c) contacting said compound with said receptor under conditions that allow said compound to bind to said receptor; and
d) determining whether said compound binds to said receptor.

2. The method according to claim 1, wherein in step a), the data provided is structural coordinate data derived from said Nuclear Overhauser Effect NMR data.

3. A method of identifying intermolecular interactions between a receptor and a known ligand of the receptor comprising the steps of:
a) contacting said receptor incorporated into a virus-like particle with said known ligand of the receptor under conditions that allow the formation of a complex between said ligand and said receptor;
b) subjecting said complex to NMR to generate Nuclear Overhauser Effect data;
c) optionally converting said Nuclear Overhauser Effect data to structural coordinate data or to bond angle data; and
d) interpreting said Nuclear Overhauser Effect data or said optionally converted Nuclear Overhauser Effect data so as to identify intermolecular interactions between the receptor and the ligand in said complex.

4. A computer for producing a three-dimensional representation of a compound bound to a receptor comprising:
a) a machine-readable data storage medium comprising a data storage material encoded with machine-readable data, wherein said data comprises Nuclear Overhauser Effect data or data derived therefrom obtained by subjecting a complex comprising a receptor incorporated into a virus-like particle and a compound bound to said receptor to NMR;
b) a working memory for storing instructions for processing said machine-readable data;
c) a central-processing unit coupled to said working memory and to said machine-readable data storage medium for processing said machine readable data into said three-dimensional representation; and
d) a display coupled to said central-processing unit for displaying said three-dimensional representation.

5. A method for evaluating the potential of a chemical entity to associate with a membrane-bound receptor comprising the steps of:
a) employing computational means to perform a fitting operation between the chemical entity and the receptor utilizing Nuclear Overhauser Effect NMR data or data derived therefrom obtained from a complex comprising a receptor incorporated into a virus-like particle and a compound bound to said receptor; and
b) analyzing the results of said fitting operation to quantify the association between the chemical entity and the receptor.

6. The method according to claim 5, comprising the additional steps of:
c) repeating steps a) and b) on a series of different chemical entities;
d) selecting two or more chemical entities that:
i) are determined to have the potential to bind to the receptor based upon the analysis of the fitting operations; and
ii) are capable of being chemically bound to one another directly or through a linker moiety;
e) contacting a compound that comprises the chemical entities selected in step d) chemically bound to one another directly or through a linker moiety with said receptor under conditions that allow said compound to bind to said receptor; and
f) determining whether said compound binds to said receptor.
